(19) United States
(12) Patent Application Publication
(10) Pub. No.: US 2026/0083576 A1
(43) Pub. Date: Mar. 26, 2026

(54) TRANSRADIAL PROSTHESIS ASSESSMENT SYSTEM

(71) Applicant: HOLLAND BLOORVIEW KIDS REHABILITATION HOSPITAL, Toronto (CA)

(72) Inventors: Calvin C. NGAN, Toronto (CA); Elaine OUELLETTE, Toronto (CA); Neil READY, Toronto (CA); Jan ANDRYSEK, Toonto (CA)

(21) Appl. No.: 19/277,985

(22) Filed: Jul. 23, 2025

Related U.S. Application Data
(60) Provisional application No. 63/698,597, filed on Sep. 25, 2024.
(60) Provisional application No. 63/825,967, filed on Jun. 18, 2025.

(51) Int. Cl. A61F 2/76; A61F 2/50; A61F 2/58; B33Y 80/00

(52) U.S. Cl. CPC A61F 2/76; A61F 2/5046; A61F 2/585; B33Y 80/00; A61F 2002/502; A61F 2002/5039; A61F 2002/5083; A61F 2002/7685; A61F 2002/7695

(57) ABSTRACT

A transradial prosthesis assessment system apparatus and method for simulating and evaluating alignment and functionality of a definitive prosthesis system for a user's diagnostic socket is provided. The apparatus includes socket adaptors, a connector, shaft housings of different sizes, extension shafts of different lengths and wrist adaptors that can be connected through different combinations to provide length and alignment adjustability to emulate the definitive prosthesis.

### FIELD

Embodiments of the present disclosure generally relate to the field of transradial prosthesis design and more specifically, embodiments relate to devices, systems and methods for improved assessment and evaluation of socket fit and prosthetic function during diagnostic socket fitting.

### INTRODUCTION

For individuals with upper-limb absence, a prosthesis serves as an artificial limb designed to restore the functions of the missing extremity. The effectiveness of a prosthesis plays a pivotal role in a person's participation in occupational, social, and daily living activities, enabling independence and enhancing the quality of life. Specifically, a prosthesis must provide effective function and control, be comfortable to wear and use, and address the specific user's cosmetic needs. Failure to meet these criteria can result in disuse and high abandonment rates, with reported levels of up to 40% across all types of upper-limb prosthesis due to comfort and function issues.

Fabricating a prosthetic device is a complex, multistep process, that hinges on several factors, such as the goals and physical presentation of the patient, and available technologies and funding. Fabricating a prosthetic device is specific to individual patients. Careful consideration must be given to the design of the prosthesis, including to the design of the prosthetic interface, also known as the socket, the selection of appropriate terminal attachments (e.g., hand, grippers), methods of control (e.g., myoelectric, body powered), and cosmetic finishes. Certain steps are typically taken to assess and check aspects of the design. For example, a diagnostic (or check) socket is commonly fabricated to evaluate and optimize suspension and comfort prior to the fabrication of the definitive prosthesis. In the case of a transradial (below elbow) prosthesis, the determination of socket fit is predicated upon prosthetists' visual examination of the residual limb in the socket (i.e. for blanching of the skin and tissue bulging), coupled with the user-reported feedback regarding comfort and function. Prosthetists then modify the shape of the socket until the user is satisfied with the design. However, assessing the diagnostic socket fit without the rest of the prosthesis, is suboptimal since comfort and prosthetic control during function tasks cannot be appropriately evaluated. The primary reasons for prosthetic device disuse, especially for myoelectric prostheses, are related to issues with comfort and function.

Further “temporary” testing prosthesis do not serve as diagnostic or simulation prosthetic systems in clinical practice. These systems cannot be used with the patient's socket, and therefore, do not solve the problem of leaving the impact of the socket-residual limb interface (socket fit) on prosthetic function largely unassessed during the diagnostic socket fitting phase. As such, within clinical practice, there is limited opportunity to assess key aspects of fit and function, as well as prosthesis alignment, prior to the fabrication of a definitive device prosthesis. Once a definitive prosthesis is made, extensive modifications are not possible, and making a new definitive prosthesis is typically time-, cost-, and resource-prohibitive. In transradial prosthetic care, current clinical practice generally lacks a viable assessment tool for evaluating socket fit and prosthetic function during diagnostic socket fitting. There is a need for a tool or apparatus that can simulate the sensation and functions of the definitive prosthesis, including the comfort and fit of the prosthetic socket, for patients, or at least an alternative.

### SUMMARY

Embodiments described herein provide a transradial prosthesis assessment system (TPAS) apparatus and method for simulating and evaluating alignment and functionality of a definitive prosthesis system for a user's diagnostic socket. The apparatus can include socket adaptors, a connector, shaft housings of different sizes, extension shafts of different lengths and wrist adaptors that can be connected through different combinations to provide length and alignment adjustability to emulate the definitive prosthesis.

The TPAS provides a modular and configurable design that interfaces with a diagnostic socket and offers length and alignment adjustability to emulate the definitive prosthesis. Its modularity facilitates setup for passive, body-powered, and myoelectric prostheses, enabling patients to simulate prosthetic function during socket fitting assessments. The TPAS is designed to be compatible with diagnostic sockets. Fitting users with a diagnostic socket is a critical step in the clinical workflow, and without assessing and achieving proper socket fit and comfort, the prosthesis is likely to be abandoned. Using the TPAS allows users to be fitted with a diagnostic socket to assess fit and comfort while also assessing alignment and function as it replicates a setup that closely represents the final definitive prosthesis.

The TPAS can be integrated with the user's diagnostic socket, facilitating socket fit evaluation and prosthesis function simulation concurrently. The features of the TPAS allow alignment adjustments at both the socket and the wrist (forearm) positions. Proper forearm alignment is essential for body symmetry and the effectiveness of the prosthesis. Furthermore, the interface configuration between the socket and the TPAS tool can be adapted to diverse socket shapes, accounting for variations in residual limb morphology and size among users. This is especially important in clinical settings where users include children and adults with congenital and acquired limb absences.

Example advantages of the embodiments described herein are improved clinical outcomes, including better designed prostheses and enhanced patient experience. Embodiments described herein also provide the advantage of improved clinical efficiency by facilitating better assessment of socket fit and prosthesis function during diagnostic socket fitting, avoiding the need for additional clinic visits, prosthesis remakes and increasing efficiency and effectiveness in prosthesis production.

Embodiments described herein present a novel apparatus that facilitates evaluation of both socket fit and prosthesis function during the diagnostic socket fit assessment. Embodiments described herein describe a clinically viable assessment tool for the fabrication of a transradial prosthesis and offer versatility to accommodate small children to large adults, different types of prostheses and terminal devices, along with ease of use for prosthetists.

In accordance with an aspect, there is provided a transradial prosthesis assessment apparatus for simulating and evaluating alignment and functionality of a definitive prosthesis system for a user's diagnostic socket, the apparatus including: socket adaptors configured to be compatible with diagnostic sockets; a connector for housing a socket adaptor, where the socket adaptor is configured for axial rotation within the connector to enable alignment adjustments; different shaft housings of different sizes, where a shaft housing is configured to connect to the connector at a hinge joint that allows for uniplanar rotation to enable alignment adjustments; different extension shafts of different lengths, where a fastener can secure an extension shaft within the shaft housing for stability; and wrist adaptors, where a wrist adaptor can attach to the extension shaft to accept the desired terminal device; where different combinations of the shaft housings and the extension shafts result in different lengths in a range between 85 mm and 260 mm.

In some embodiments, the connector, shaft housing, extension shaft and wrist adaptor can be made with different materials. For example, the connector, shaft housing, extension shaft and wrist adaptor can comprise one or more metal alloys. For example, the connector, shaft housing, extension shaft and wrist adaptor can comprise aluminum. This may help with cost and machinability. As another example, the connector, shaft housing, extension shaft and wrist adaptor can comprise stainless steel.

In some embodiments, the socket adaptor can be created using different methods and techniques. For example, in some embodiments, the socket adaptor can be created using fused deposition modelling 3D printing. As another example, in some embodiments, the socket adaptor could be injection moulded.

In some embodiments, fasteners (e.g. bolted joints) are used to secure the wrist adaptor and extension shaft.

In some embodiments, the shaft housing comprises a clamp ring for stability.

In some embodiments, the socket adaptor is single-use.

In some embodiments, the fastener is designed to be compatible with a 5.0 mm Allen key wrench.

In some embodiments, the apparatus supports use in passive, body-powered and myoelectric prosthetic systems.

In accordance with an aspect, there is provided an adjustable transradial prosthesis assessment apparatus for simulating and evaluating alignment and functionality of a definitive prosthesis system for a user's diagnostic socket, the apparatus including: a socket adaptor configured to connect to a user's diagnostic socket, where the socket adaptor is selected from socket adaptors each configured to be compatible with diagnostic sockets; a connector for housing the socket adaptor, where the socket adaptor is configured for axial rotation within the connector to enable alignment adjustments; a shaft housing connected to the connector at a hinge joint that allows for uniplanar rotation to enable alignment adjustments, where the shaft housing is selected from different shaft housings of different sizes; an extension shaft, where the extension shaft is selected from different extension shafts of different lengths, where a fastener secures the extension shaft to the shaft housing for stability; and a wrist adaptor attached to the extension shaft; where the shaft housing together and the extension shaft provide an adjustable length of the apparatus between a range of 85 mm and 260 mm.

In some embodiments, the weight of the apparatus ranges between 212 g and 373 g.

In some embodiments, the connector, shaft housing, extension shaft and wrist adaptor can be made with different materials. For example, the connector, shaft housing, extension shaft and wrist adaptor can comprise one or more metal alloys. For example, the connector, shaft housing, extension shaft and wrist adaptor can comprise aluminum. As another example, the connector, shaft housing, extension shaft and wrist adaptor can comprise stainless steel.

In some embodiments, the socket adaptor can be created using different methods and techniques. For example, in some embodiments, the socket adaptor can be created using fused deposition modelling 3D printing. As another example, in some embodiments, the socket adaptor can be made using injection moulding.

In some embodiments, fasteners secure the wrist adaptor and extension shaft. In some embodiments, bolted joints secure the wrist adaptor and extension shaft.

In some embodiments, the shaft housing comprises a clamp ring for stability.

In some embodiments, the socket adaptor is single-use.

In some embodiments, the fastener is designed to be compatible with a 5.0 mm Allen key wrench.

In some embodiments, the apparatus supports use in passive, body-powered and myoelectric prosthetic systems.

In some embodiments, the socket adaptor connects to the user's diagnostic socket using a fast-curing adhesive.

In accordance with an aspect, there is provided a method for an adjustable transradial prosthesis assessment apparatus for simulating and evaluating alignment and functionality of a definitive prosthesis system for a user's diagnostic socket, the method including: selecting a socket adaptor from socket adaptors each configured to be compatible with diagnostic sockets, where a connector is configured for housing the socket adaptor, where the socket adaptor is configured for axial rotation within the connector to enable alignment adjustment; selecting a shaft housing from different shaft housings of different sizes, the shaft housing configured to connect to the connector at a hinge joint that allows for uniplanar rotation to enable alignment adjustments; selecting an extension shaft from extension shafts of different lengths, where a fastener secures the extension shaft to the shaft housing for stability; and selecting a wrist adaptor from wrist adaptors configured to attach to the extension shaft; where the shaft housing together and the extension shaft provide an adjustable length of the apparatus between a range of 85 mm and 260 mm.

### DESCRIPTION OF THE FIGURES

In the figures, embodiments are illustrated by way of example. It is to be expressly understood that the description and figures are only for the purpose of illustration and as an aid to understanding.

Embodiments will now be described, by way of example only, with reference to the attached figures, wherein in the figures:

FIG. 1 is an example diagram displaying the five major types of components of the TPAS, according to some embodiments.

FIG. 2 is a diagram of an example assembly of the TPAS components, according to some embodiments.

FIG. 3 is a diagram of example alignment adjustments for the TPAS that can be attained at two levels, according to some embodiments.

FIG. 4 is a photo showing an example practice of evaluating socket suspension, according to some embodiments.

FIG. 5 is a photo showing example patients with passive, body-powered and myoelectric prosthesis configurations of the TPAS, according to some embodiments.

FIG. 6 is a graph comparing example Box and Blocks Test (mBBT) scores in a pilot study for patients using the proposed TPAS (myoelectric prosthesis configuration) and their definitive prosthesis, according to some embodiments.

### DETAILED DESCRIPTION

Embodiments described herein provide a transradial prosthesis assessment system (TPAS) apparatus and method for simulating and evaluating alignment and functionality of a definitive prosthesis system for a user's diagnostic socket is provided. The apparatus includes socket adaptors, a connector, shaft housings of different sizes, extension shafts of different lengths and wrist adaptors that can be connected through different combinations to provide length and alignment adjustability to emulate the definitive prosthesis.

Embodiments described herein provide a modular and configurable design that interfaces with a diagnostic socket and offers length and alignment adjustability to emulate the definitive prosthesis. The modularity facilitates setup for passive, body-powered, and myoelectric prostheses, enabling patients to simulate prosthetic function during socket fitting assessments. The TPAS is designed to be compatible with diagnostic sockets fabricated through conventional methods. Fitting users with a diagnostic socket to assess fit and comfort is a critical step in the clinical workflow, and without achieving socket comfort, the prosthesis is likely to be abandoned. Using the TPAS allows users with limb absence at the transradial (below-elbow) level to be fitted with a diagnostic socket to assess fit and comfort, which is representative of what the final prosthesis will be like.

Embodiments described herein provide that the TPAS can be integrated with the user's diagnostic socket, facilitating socket fit evaluation and prosthesis function simulation concurrently. The alignment capabilities of TPAS allow alignment adjustments at both the socket and the wrist (forearm) positions. Proper forearm alignment is essential for body symmetry and the effectiveness of the prosthesis. Furthermore, the interface configuration between the socket and the TPAS tool can be adapted to diverse socket shapes, accounting for variations in residual limb morphology and size among users. This is especially important in clinical settings where users include children and adults.

For unilateral transradial prosthetic users, the length and alignment of their prostheses, which refer to the positioning of the wrist and terminal device relative to the socket, are crucial for optimizing prosthesis functionality and achieving symmetry with the intact side. The elbow-to-wrist distance can range from 110 to 280 mm among children (aged 2 and up) and adult able-bodied individuals, with a recommended shortest functional transradial residual limb length of at least 20 mm. In some embodiments, the TPAS allows for an adjustable length of 90 to 260 mm from the distal end of the socket to the wrist. In some embodiments, the TPAS allows for angle adjustments of at least 130 degrees and rotation adjustments of at least 140 degrees for the forearm (i.e., at the distal end of the socket) and the wrist. This allows the TPAS to attain a desired prosthesis alignment (orientation of terminal device relative to the socket in three planes).

Prosthetic needs are highly individualized, leading to the prescription of different types of devices, including passive, body-powered, and myoelectric prostheses. Embodiments described herein provide a design that is compatible with existing upper-limb terminal devices, allowing the use of standardized prosthetic wrist units for all three types of prosthesis for both adult and paediatric populations.

In some embodiments, all fasteners within the design feature the same drive style and driver size for ease of use, such that all adjustments can be made with a single tool.

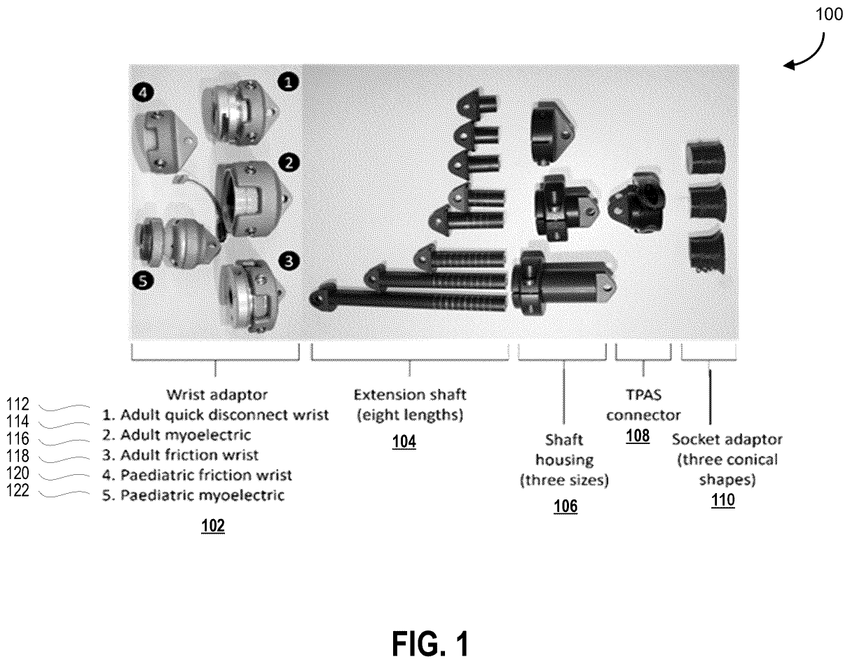

FIG. 1 is an example diagram 100 displaying the five major types of components of the TPAS, according to some embodiments.

The TPAS functions as a “temporary” testing prosthesis, where a prosthetist can fit the TPAS onto the patient's diagnostic socket during a socket fitting assessment. This allows prosthetists to evaluate the fit, alignment, and functionality of the prosthetic system in real time. The TPAS interfaces with the patient's diagnostic socket and offers length and alignment adjustability to represent the definitive prosthesis. Furthermore, its modularity facilitates setup for passive, body-powered and myoelectric prostheses, enabling patients to simulate prosthetic function during their socket fitting assessment.

The TPAS provides a modular and configurable design comprising five major components: a socket adaptor 110, a connector 108, a shaft housing 106, an extension shaft 104, and a wrist adaptor 102. The transradial diagnostic socket is connected to the TPAS through the socket adaptor 110. The socket adaptor 110 is housed within the TPAS connector 108, which is then connected to the shaft housing 106. The extension shaft 104 is attached to the wrist adaptor 102 and fastened within the shaft housing 106 by a fastener to ensure stability. In some embodiments, as in FIG. 1, the components are colour-coded to discern compatible parts for greater ease of use.

In some embodiments, there are three different conical shapes of socket adaptors for the user to select from to accommodate different socket shapes. Due to variations in the shapes and sizes of residual limbs, socket adaptors with different conical shapes (i.e., different opening diameters) can be used to accommodate them. Before securing the socket adaptor to the distal end of the socket, the prosthetist can perform a dry fit to determine which adaptor best contours to the distal end. Once selected, the prosthetist fills the cup of the socket adaptor with epoxy resin and glues it to the socket.

In some embodiments, the socket adaptor 110 is a single-use component made via fused deposition modelling 3D printing.

In some embodiments, there are three different sizes of shaft housings for the user to select from.

In some embodiments, there are eight different lengths of extension shafts for the user to select from. Due to variations in the shapes and sizes of residual limbs, shaft housings and extension shafts of different lengths and sizes can be used to accommodate them. The shaft housings and extension shafts can be used together and can be colour-coded (e.g. red, blue, and green) to ensure proper pairing; for instance, green shafts should be used with green housings. This system ensures minimal shaft engagement inside the housing, allowing the shaft to be securely tightened and reducing the risk of it coming loose during use. With three shaft housings and eight extension shafts, a total of eight possible pairings (and combinations) enables length adjustments ranging from 85 mm to 260 mm. The length of the TPAS is determined during the clinical assessment, considering factors such as function, prosthesis purpose, and body symmetry. Based on collective input, the TPAS length can be adjusted until the patient achieves the desired functional performance and is satisfied with the results.

In some embodiments, there are five different types of wrist adaptors for the user to select from: adult quick disconnect wrist, adult myoelectric wrist, adult friction wrist, paediatric friction wrist, and paediatric myoelectric wrist.

In some embodiments, the extension shaft 104 and the wrist adaptor 102 are secured by fasteners (e.g. bolted joints). In some embodiments, a clamp ring fastens the extension shaft 104 within the shaft housing 106.

In some embodiments, all components (except for the socket adaptor 110) are designed to be machined from aluminum, which provides durability and structural integrity for clinical applications.

In some embodiments, the TPAS can be assembled with different combinations of its components such that the weight of the TPAS (excluding the terminal device) ranges from 212 g to 373 g.

In some embodiments, all fasteners, including the socket head screws and set screws, utilize the 5.0 mm Allen key wrench size.

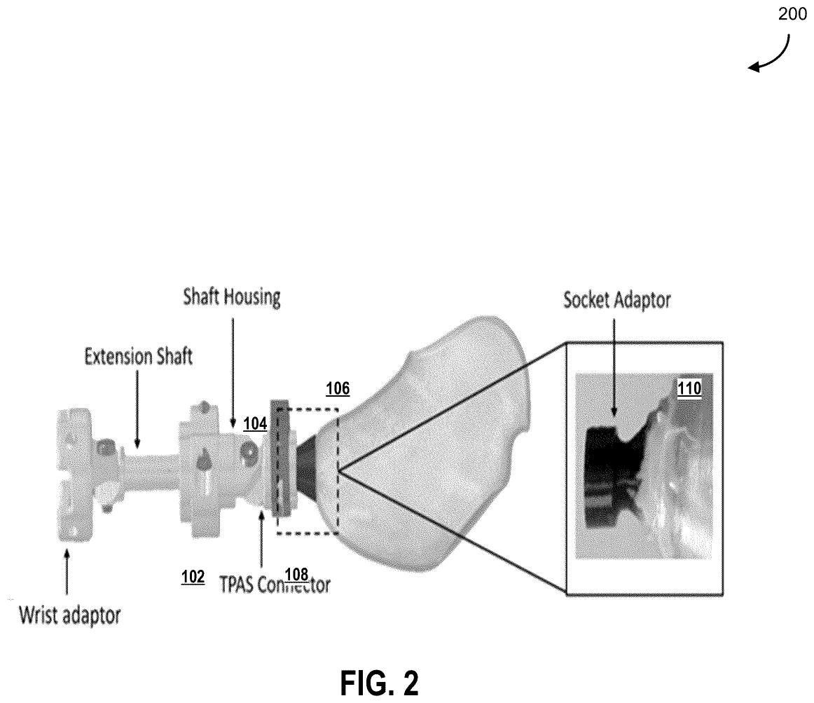

FIG. 2 is a diagram of an example assembly of the TPAS components, according to some embodiments.

Prior to using the TPAS, prosthetists can first mark the midlines of the socket on both lateral and anterior sides. Subsequently, the socket adaptor 110 of the TPAS can be attached to the distal end of the socket at the intersection of both midlines. In some embodiments, a rapid curing and high strength adhesive is used to attach the socket adaptor 110 to the socket. As shown in FIG. 2, the diagnostic socket (along with the socket adaptor 110) is then attached to the rest of the TPAS through the TPAS connector 108.

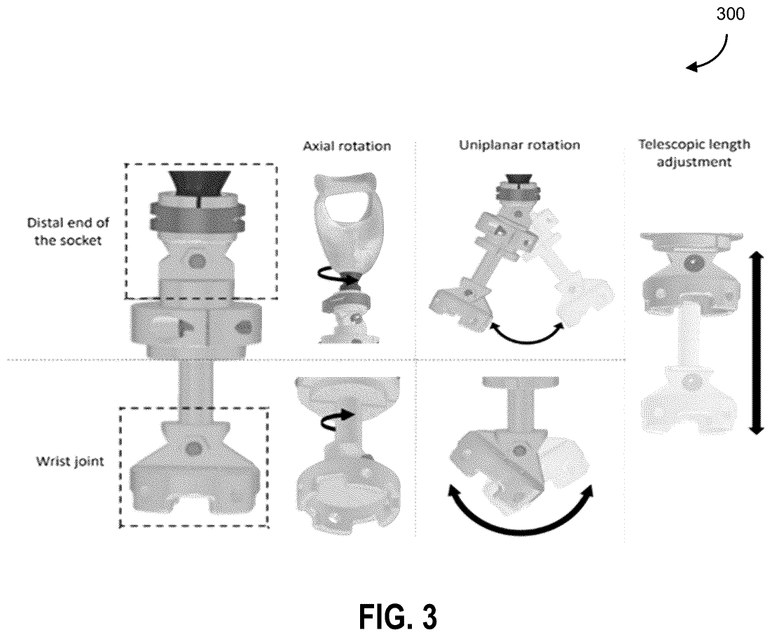

FIG. 3 is a diagram of example alignment adjustments for the TPAS that can be attained at two levels, according to some embodiments.

The design of the TPAS features length and alignment adjustments. Embodiments described herein provide a telescopic design, which involves a combination of the shaft housing and the extension shaft to enable length adjustments. The different sizes available for the shaft housings and the different lengths available for the extension shafts can be utilized to make different combinations of a shaft housing with an extension shaft. In some embodiments, combinations of these components yield a length ranging from 85 mm to 260 mm from the distal end of the socket to the wrist.

As shown in FIG. 3, the overall alignment adjustment of the TPAS can be adjusted at two levels: the distal end of the socket and the wrist joint. Each level provides two degrees of freedom in alignment—axial rotation along the longitudinal axis and uniplanar rotation at the hinge joint.

First, at the distal end of the socket, the socket adaptor can rotate axially within the TPAS connector. The hinge joint between the TPAS connector and the shaft housing also allows for uniplanar rotation. This axial-uniplanar rotation combination enables the TPAS alignment from the socket to the wrist joint (i.e., the forearm). Second, to refine the position of the terminal device, adjustments can be made at the wrist level. Axial rotation is achieved by rotating the extension shaft within the shaft housing, while a hinge joint at the distal end of the extension shaft permits planar rotation adjustments for the terminal device.

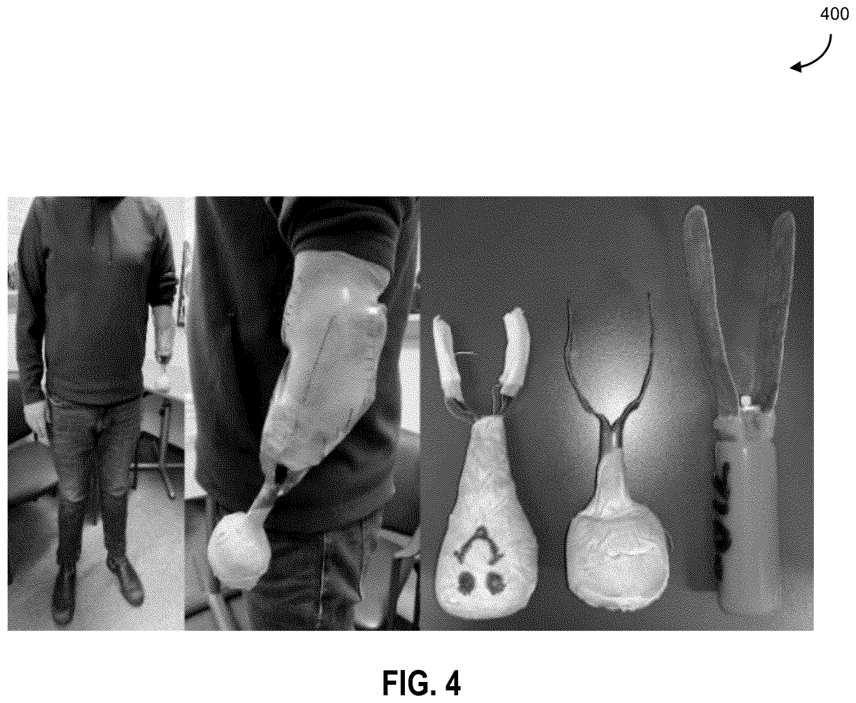

FIG. 4 is a photo showing an example practice of evaluating socket suspension, according to some embodiments.

The practice for evaluating socket suspension and comfort in FIG. 4 involves prosthetists affixing a weight by taping the struts to the diagnostic socket to emulate the weight characteristics of the definitive prosthesis. Depending on the age of the patient and their prescribed prosthesis type, there can be different weight options (e.g., 400 g, 615 g, and 708 g) available, where a lighter weight (e.g., 400 g) is typically designated for paediatric users. To be inclusive of paediatric users, the weight of the TPAS (excluding the terminal device) is designed to not exceed 400 g.

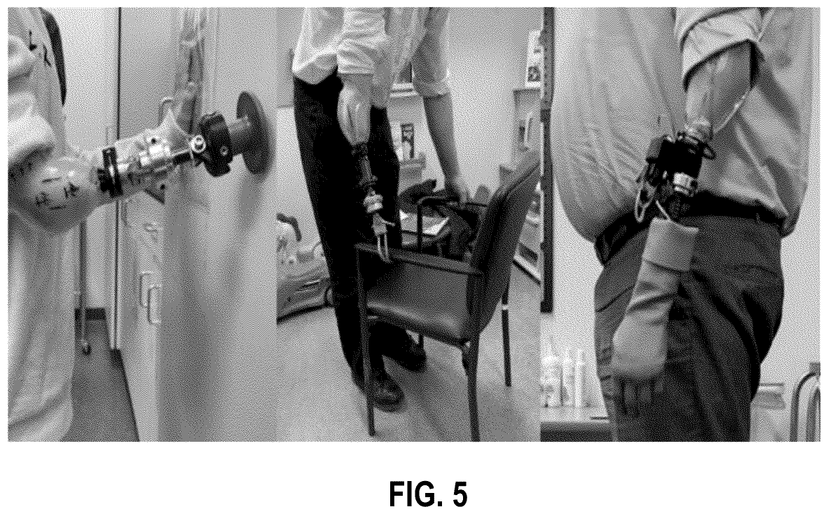

FIG. 5 is a photo showing example patients with passive, body-powered and myoelectric prosthesis configurations of the TPAS, according to some embodiments.

With its unique modular architecture, the TPAS can be configured with three distinct types of terminal devices—passive, body-powered, and myoelectric. Each terminal device interfaces with its own respective wrist unit. In some embodiments, the wrist adaptors shown in FIG. 1 are used to accommodate selected commercially available wrist units. The wrist adaptors in FIG. 1 are some of the most commonly used components in hospital clinical practice. The wrist adaptor can be attached to the distal end of the extension shaft through a fastener, such as a bolted joint.

In some embodiments, the TPAS can be configured into a passive prosthesis by attaching a passive terminal device using the wrist adaptors designed for passive wrist units (adult or paediatric friction wrist).

In some embodiments, the TPAS can accommodate a body-powered terminal device with the adult quick disconnect wrist.

In some embodiments, the TPAS can be configured for a myoelectric terminal device, using both the appropriate wrist adaptor (e.g., adult or paediatric myoelectric) and the electronics of the myoelectric system. Myoelectric prosthesis control is contingent on the electromyographic signals acquired through the surface electrodes placed over voluntary muscle groups within the residual limb. The initial step involves identifying suitable electrode sites on the prosthesis user's limb. The prosthetist then modifies the diagnostic socket to mount the electrodes at the specified sites. Subsequently, the electronics, including the electrodes, controller, and batteries, are connected to the wrist unit via cables. In some embodiments, these components are stored in a custom-made pouch that is attached to the TPAS using Velcro.

In an illustrative example study, individuals with an upper limb absence at the transradial level were recruited to participate in a comparative trial between the proposed TPAS and a conventional method used in hospitals. Inclusion criteria for this example study were as follows: 1) had a transradial limb absence for at least 2 years and 2) needed a new prosthesis, including a socket, at the time of data collection. The sample of convenience involved patients accessing services at a hospital for a new transradial prosthesis. Informed consent was obtained from all participating patients involved in the study. Each patient attended a data collection session during the diagnostic socket fitting assessment. Those who were prescribed with a myoelectric prosthesis attended an additional session during the definitive prosthesis provision.

The comparative, open example trial was conducted to assess the effectiveness of the TPAS compared to a conventional method used in hospitals (i.e., affix a weight to the socket; hereafter referred to as “CONV”) in facilitating the socket fit evaluation. Additionally, the treating prosthetists were asked to evaluate the usability of the TPAS. Relevant health information, such as sex, age, and cause of limb absence were collected.

Mirroring conventional practice, during the diagnostic socket fitting assessment, the diagnostic socket was first fitted onto the patient and evaluated for fit, suspension, and comfort. Adjustments were made to the diagnostic socket by the treating prosthetist to improve socket fit and comfort, as needed, and repeated until the patient was satisfied.

Subsequently, each patient was fitted successively with the CONV and TPAS onto their diagnostic socket in a randomized order, determined by computer-generated random numbers. Guided by the treating prosthetist and an attending occupational therapist, the patient underwent a standardized clinical assessment after each fitting, performing common arm movements such as abduction, adduction, flexion, and extension. The purpose was to further examine socket fit and comfort. The weight was secured by taping the struts onto the socket. The TPAS was set up in accordance with the patient's prescribed prosthesis type—passive, body-powered, or myoelectric, including the corresponding terminal device. The treating prosthetist then adjusted the length and alignment of the TPAS, considering the patient's intact limb based on visual examination, physical measurement, and patient feedback, as is common clinical practice.

After completing clinical assessments with both the CONV and TPAS, each patient was given the Quebec User Evaluation of Satisfaction with Assistive Technology 2.0 (QUEST) questionnaire to assess each device separately. Specifically, the first eight items related to the technology on the questionnaire were used. Patients were asked to evaluate their satisfaction on various aspects of each device, including weight, dimensions, and effectiveness, using a scale from 1 (not satisfied at all) to 5 (very satisfied). The QUEST scores for both devices were computed by adding the ratings of all valid responses, then dividing the sum by the number of valid items. The System Usability Scale (SUS) was also administered to the treating prosthetist to assess their perceived usability of both the CONV and TPAS. The goal was to assess the feasibility of implementing TPAS into their clinical workflow. The SUS comprises ten items, each rated on a 5-point Likert scale, from 1 (strongly disagree) to 5 (strongly agree). Prosthetists were asked to evaluate the usability of the TPAS and CONV separately. SUS scores for both devices were calculated following the instructions provided, where the final score of each item ranged from 0 to 4, with the total score ranging from 0 to 100.

Descriptive statistics, including the mean and standard deviation values, were calculated for the QUEST and SUS outcome measures. Normality of the data for the overall QUEST and SUS scores was assessed using the Shapiro-Wilk test. Significant differences between the CONV and TPAS overall scores were determined using a paired samples t-test if there was no evidence of non-normality; otherwise, a non-parametric test (Wilcoxon signed-rank test) was used. P-values less than 0.05 were considered statistically significant. To determine the effect size, Cohen's D was calculated for the paired samples t-test, with effect size classified as small (0.2), medium (0.5), and large (>0.8). For the Wilcoxon signed-rank test, effect size was determined by the matched rank biserial correlation, classified as small (0.1), medium (0.3), large (0.5), and very large (0.7). All statistical analyses were performed using an open-source program, JASP 0.17.3.0.

To assess the effectiveness of the TPAS in enabling patients to simulate myoelectric prosthesis control during diagnostic socket fitting, the modified Box and Blocks Test was administered to those prescribed with a myoelectric prosthesis (n=3). It is a functional test that measures unilateral gross manual dexterity. Patients were asked to complete the test twice: 1) with the TPAS during the diagnostic socket fitting assessment and 2) with their definitive prosthesis at the time of the prosthesis provision.

The test involved a box measuring 53.7 cm in length and 25.4 cm width, with a 15.2 cm tall partition inserted to divide the box lengthwise into two equal-sized compartments. Sixteen 2.5 mm cubic blocks were arranged in 4 rows within the compartment on the same side as the prosthetic limb. Patients were seated on a standard height chair in front of the box. They were instructed to transport and drop each block on the other side of the partition following a specific pattern-starting with the block farthest away from the partition and the patient, then across the row to the block closest to the partition, then down to the next row from the outside block inward. Patients were required to ensure that the fingertip crossed the partition plane each time. A 15-second practice period preceded the testing. The time taken to complete moving all 16 blocks or the number of blocks moved within 1 minute, whichever the patient achieved first, was recorded. If a patient successfully moved all 16 blocks within 1 minute, their score was extrapolated to represent a full minute. For example, if a patient moved all 16 blocks in 30 seconds, then their score would be 32.

Table 1 shows the results of the pilot study for the recruited patients (n=7). The first column of the table indicates the participants.

TABLE I
PATIENT CHARACTERISTICS

| | | | | | | Years Using |
|---|---|---|---|---|---|---|
| | | Cause | | | | the Same |
| | | of limb | | | Prosthesis | Type of |
| | Sex | absence | Side | Age | Prescribed | Prosthesis |
| 1 | M | Congenital | L | 22 | Body- | 0 |
| | | | | | powered | |
| 2 | M | Congenital | L | 51 | Passive | 50 |
| 3 | M | Congenital | L | 36 | Myoelectric | 12 |
| 4 | F | Acquired | R | 8 | Myoelectric | 0 |
| 5 | M | Congenital | L | 60 | Body- | 55 |
| | | | | | powered | |
| 6 | M | Acquired | R | 16 | Body- | 8 |
| | | | | | powered | |
| 7 | M | Congenital | L | 9 | Myoelectric | 0 |

Table 2 shows the weight measurements of the patients' CONV, TPAS, and definitive prostheses. The first column of the table indicates the participants.

TABLE II
WEIGHT OF THE CONV, TPAS,
AND DEFINITIVE PROSTHESIS

| | CONV (g) | TPAS (g) | Definitive Prosthesis (g) |
|---|---|---|---|
| 1 | 710 | 652 | 1019 |
| 2 | 775 | 763 | 851 |
| 3 | 1061 | 1338 | 1322 |
| 4 | 489 | 345 | 454 |
| 5 | 697 | 539 | 600 |
| 6 | 799 | 692 | 721 |
| 7 | 659 | 740 | 757 |

Socket and terminal device are included in the measurement.

Table 3 shows the results of the QUEST scores from the patients. The first column of the table indicates the participants.

TABLE III
QUEST SCORE

| | Item Content | CONV | TPAS |
|---|---|---|---|
| 1 | The dimensions (size, height, length, | 2.6 (0.8) | 4.6 (0.5) |
| | width) of the technology? | | |
| 2 | The weight of the technology? | 2.1 (0.7) | 4.0 (1.0) |
| 3 | The ease in adjusting (fixing, fastening) | 2.6 (1.0) | 4.6 (0.5) |
| | the parts of the technology? | | |
| 4 | How safe and secure the technology is? | 2.6 (1.0) | 4.6 (0.5) |
| 5 | The durability (endurance, resistance to | 3.0 (1.4) | 4.7 (0.5) |
| | wear) of the technology? | | |
| 6 | How easy it is to use the technology? | 2.7 (1.3) | 4.6 (0.5) |
| 7 | How comfortable the technology is? | 2.8 (1.3) | 4.6 (0.8) |
| 8 | How effective the technology is (the | 3.1 (1.3) | 4.7 (0.5) |
| | degree to which the technology meets | | |
| | your needs)? | | |

| | | |
|---|---|---|
| Overall Score | 2.6 (0.9) | 4.6 (0.5) |

Mean (1 standard deviation) of the patient's satisfaction (QUEST) scores. Each item and the overall score are scored out of 5. Significant difference (p = 0.004; effect size = 1.75) was observed in the overall score between CONV and TPAS.

The Shapiro-Wilk test results (p=0.262) showed no evidence of non-normality for the 5 overall score. The TPAS had significant higher overall scores compared to the CONV (4.6 vs 2.6 out of 5, p=0.004). A very large effect size (effect size=1.75) was also found.

Table 4 depicts the analysis of the SUS scores from the prosthetists.

TABLE IV
SUS SCORE

| | Item Content | CONV | | TPAS | |
|---|---|---|---|---|---|
| 1 | I think that I would like to use this | 1.1 | (0.9) | 3.7 | (0.5) |
| | system frequently | | | | |
| 2 | I found the system unnecessarily | 0.0 | (0.0) | 0.7 | (0.5) |
| | complex | | | | |
| 3 | I thought the system was easy to use | 2.7 | (0.8) | 3.0 | (0.0) |
| 4 | I think that I would need support of a | 0.0 | (0.0) | 0.1 | (0.4) |
| | technical person to be able to use this | | | | |
| | system | | | | |
| 5 | I found that the various functions in this | 0.0 | (0.0) | 3.1 | (0.4) |
| | system were well integrated | | | | |
| 6 | I thought that there was too much | 0.6 | (1.1) | 0.1 | (0.4) |
| | inconsistency in this system | | | | |
| 7 | I would imagine the most people learn | 3.8 | (0.4) | 3.0 | (0.0) |
| | to use this system very quickly | | | | |
| 8 | I found the system very cumbersome to | 1.1 | (0.4) | 0.7 | (0.5) |
| | use | | | | |
| 9 | I felt very confident using the system | 3.6 | (1.1) | 3.3 | (0.5) |
| 10 | I needed to learn a lot of things before I | 0.0 | (0.0) | 0.7 | (0.5) |
| | could get going with this system | | | | |

| | | | | |
|---|---|---|---|---|
| Overall Score | 73.9 | (9.6) | 84.3 | (3.5) |

Mean (1 standard deviation) of the system usability scale (SUS) scores. Each item is scored out of 4, ranging from strongly disagree (0) to strongly agree (4). The overall score is out of 100. Significant difference (p = 0.02; effect size = 1.00) was observed in the overall SUS score between CONV and TPAS.

Three (n=3) prosthetists were recruited. The overall SUS score showed significant deviation from normality (p<0.001) according to the Shapiro-Wilk test. Significantly higher SUS score was found for the TPAS, (84.3 vs 79.9 out of 100, p=0.02), corresponding to a very large effect size (effect size=1.00). Prosthetists favoured the TPAS regarding preference for frequent use and the integration of the system's function. In contrast, CONV was perceived as quicker for people to learn.

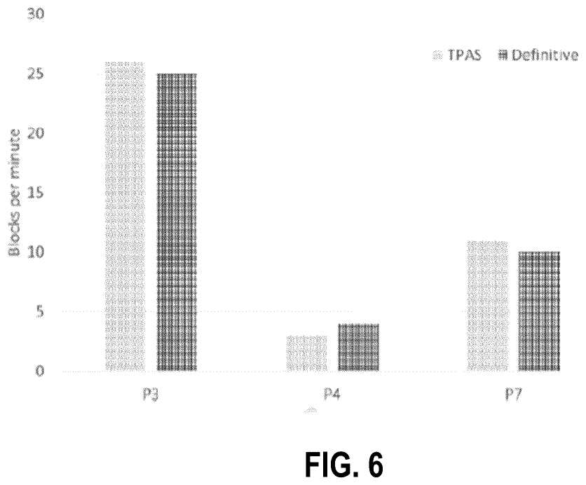

FIG. 6 is a graph comparing example Box and Blocks Test (mBBT) scores for patients using the proposed TPAS (myoelectric prosthesis configuration) and their definitive prosthesis, according to some embodiments.

Three (n=3) participants prescribed with the myoelectric prosthesis performed the modified Box and Blocks Test (mBBT). Trends observed from FIG. 6 indicated similar performances were achieved with both the TPAS and definitive prostheses.

Embodiments described herein present a novel device that facilitates evaluation of both socket fit and prosthesis function during the diagnostic socket fit assessment. Embodiments described herein describe a clinically viable assessment tool for the fabrication of a transradial prosthesis and offer versatility to accommodate small children to large adults, different types of prostheses and terminal devices, along with ease of use for prosthetists. The acceptance and sustained use of a prosthetic device depends on its alignment with user expectations and needs, and achieving an appropriate prosthetic design is crucial. This includes optimizing the socket fit, but also the composition and setup of the rest of the prosthesis.

The higher overall QUEST score in the pilot study results revealed a significantly stronger satisfaction level among patients with the TPAS design. Moreover, patients expressed higher satisfactions in all categories, strongly indicating the effectiveness of the TPAS design. In particular, they valued the TPAS's capability to replicate a setup that closely represents their definitive prosthesis. For example, patients were able to test various activity-specific terminal devices for their passive recreational prostheses, including a shroom tumbler for floor exercises and a handlebar adaptor for biking. The TPAS was especially useful for patients with limited experience using myoelectric prostheses. They were able to start practicing and familiarizing with the control mechanisms with their occupational therapists during the diagnostic socket fitting appointment, rather than awaiting the fabrication of the definitive prostheses. This also allowed their treating prosthetists and occupational therapists to observe and optimize the socket and setups for the prostheses concurrently. Although only three patients were fitted with myoelectric prostheses and performed the mBBT in the study, the initial results seemed to suggest that the TPAS showed promise in assessing aspects of prosthetic function comparable to a definitive prosthesis. The results of the study indicate that TPAS was effective in enhancing the patient's clinical experience compared to the conventional practice during diagnostic socket fitting assessment.

When comparing the usability between CONV and TPAS from the prosthetists' perspective, significant difference was also found in the SUS scores. On an item-per-item comparison, the CONV was rated better compared to the TPAS regarding how quickly most people can learn to use the system. This is because the CONV simply involves prosthetists affixing a weight to the diagnostic socket with adhesive tape, which is simpler than the TPAS set up. However, the TPAS received higher ratings in terms of the perceived integration of its functions. This can be attributed to the TPAS's versatility to simulate many prosthetic functions. Therefore, a trade-off exists between a system like CONV that might be simpler to setup, and one like the TPAS, which can more comprehensively test aspects of prosthetic fit and function. Ultimately, user acceptance is the most critical aspect when evaluating the effectiveness of the design, as overly complex systems often go unused, thereby failing to fulfill the intended purpose.

In some embodiments, the TPAS further incorporates a baseplate and/or retainer for a cable system to facilitate operational control of body-powered prostheses. Body-powered prostheses operate using a cable system, where one end of the steel cable is connected to the terminal device (such as a prosthetic hook), while the other end is attached to a harness that is secured around the contralateral shoulder. The cable is guided by the baseplate attached to the exterior of the prosthesis.

In some embodiments, the TPAS uses a spectra cable for the TPAS setup instead of a steel cable. To maintain proper tension on the cable for the prosthesis to function effectively, the cable must be trimmed to the correct length. Each body-powered prosthesis apparatus contains only one cable, which needs to be reserved for the definitive prosthesis.

In some embodiments, the TPAS is directly integrated into the prosthesis fabrication process to aid in setting up the definite prosthesis. For instance, similarly to an alignment jig used in lower limb prosthesis fabrication, an alignment jig can be designed specifically for the TPAS. This jig would transfer the alignment information captured by the TPAS, such as the relative locations between the socket and the wrist unit, to the definitive prosthesis which is essential during the fabrication process. For example, this can be done using imaging, parametric modeling, and simple markings on the TPAS (i.e. gradients on the system to show the set angles at each joint and length of the shaft).

The findings of the pilot study suggested the TPAS was effective in enhancing the patient's clinical experience compared to the conventional practice. Embodiments described herein facilitate visualization of the prosthesis and provide a more realistic and immersive experience for the patients in understanding its control and functions. Prosthetists have also found the functions of TPAS to be well-integrated and favoured the TPAS regarding preference for frequent use over the conventional method.

Applicant notes that the described embodiments and examples are illustrative and non-limiting. Practical implementation of the features may incorporate a combination of some or all of the aspects, and features described herein should not be taken as indications of future or existing product plans. Applicant partakes in both foundational and applied research, and in some cases, the features described are developed on an exploratory basis.

The term “connected” or “coupled to” may include both direct coupling (in which two elements that are coupled to each other contact each other) and indirect coupling (in which at least one additional element is located between the two elements).

Although the embodiments have been described in detail, it should be understood that various changes, substitutions and alterations can be made herein without departing from the scope. Moreover, the scope of the present application is not intended to be limited to the particular embodiments of the process, machine, manufacture, composition of matter, means, methods and steps described in the specification.

As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

As can be understood, the examples described above and illustrated are intended to be exemplary only.

What is claimed is:

1. A transradial prosthesis assessment apparatus for simulating and evaluating alignment and functionality of a definitive prosthesis system for a user's diagnostic socket, the apparatus comprising:
a plurality of socket adaptors configured to be compatible with diagnostic sockets;
a connector for housing a socket adaptor of the plurality of socket adaptors, wherein the socket adaptor is configured for axial rotation within the connector to enable alignment adjustments;
a plurality of different shaft housings of different sizes, wherein a shaft housing of the plurality of shaft housings is configured to connect to the connector at a hinge joint that allows for uniplanar rotation to enable alignment adjustments;
a plurality of different extension shafts of different lengths, wherein a fastener can secure an extension shaft of the plurality of extension shafts within the shaft housing of the plurality of shaft housings for stability;
a plurality of wrist adaptors, wherein a wrist adaptor of the plurality of wrist adaptors can attach to the extension shaft; and
wherein different combinations of the plurality of shaft housings and the plurality of extension shafts result in different lengths in a range between 85 mm and 260 mm.

2. The apparatus of claim 1, wherein the connector, shaft housing, extension shaft and wrist adaptor comprise one or more of aluminum and stainless steel.

3. The apparatus of claim 1, wherein the socket adaptor is created using one or more of injection moulding and fused deposition modelling 3D printing.

4. The apparatus of claim 1, wherein a plurality of fasteners or bolted joints secure the wrist adaptor and extension shaft.

5. The apparatus of claim 1, where in the shaft housing comprises a clamp ring for stability.

6. The apparatus of claim 1, wherein the socket adaptor is single-use.

7. The apparatus of claim 1, wherein the fastener is designed to be compatible with a 5.0 mm Allen key wrench.

8. The apparatus of claim 1, wherein the kit supports use in passive, body-powered and myoelectric prosthetic systems.

9. An adjustable transradial prosthesis assessment apparatus for simulating and evaluating alignment and functionality of a definitive prosthesis system for a user's diagnostic socket, the apparatus comprising:
a socket adaptor configured to connect to a user's diagnostic socket, wherein the socket adaptor is selected from a plurality of socket adaptors each configured to be compatible with diagnostic sockets;
a connector for housing the socket adaptor, wherein the socket adaptor is configured for axial rotation within the connector to enable alignment adjustments;
a shaft housing connected to the connector at a hinge joint that allows for uniplanar rotation to enable alignment adjustments, wherein the shaft housing is selected from a plurality of different shaft housings of different sizes;
an extension shaft, wherein the extension shaft is selected from a plurality of extension shafts of different lengths, wherein a fastener secures the extension shaft to the shaft housing for stability; and
a wrist adaptor attached to the extension shaft;
wherein the shaft housing together and the extension shaft provide an adjustable length of the apparatus between a range of 85 mm and 260 mm.

10. The apparatus of claim 9, wherein the weight of the device ranges between 212 g and 373 g.

11. The apparatus of claim 9, wherein the connector, shaft housing, extension shaft and wrist adaptor comprise one or more metal alloys.

12. The apparatus of claim 9, wherein the socket adaptor is created using one or more of injection moulding, machining, additive manufacturing.

13. The apparatus of claim 9, wherein a plurality of fasteners secure the wrist adaptor and extension shaft.

14. The apparatus of claim 9, where in the shaft housing comprises a clamp ring for stability.

15. The apparatus of claim 9, wherein the socket adaptor is single-use.

16. The apparatus of claim 9, wherein the fastener is designed to be compatible with a 5.0 mm Allen key wrench.

17. The apparatus of claim 9, wherein the kit supports use in passive, body-powered and myoelectric prosthetic systems.

18. The apparatus of claim 9, wherein the socket adaptor connects to the user's diagnostic socket using a fast-curing adhesive.

19. A method for an adjustable transradial prosthesis assessment apparatus for simulating and evaluating alignment and functionality of a definitive prosthesis system for a user's diagnostic socket, the method comprising:
selecting a socket adaptor from a plurality of socket adaptors each configured to be compatible with diagnostic sockets, wherein a connector is configured for housing the socket adaptor, wherein the socket adaptor is configured for axial rotation within the connector to enable alignment adjustment;
selecting a shaft housing from a plurality of different shaft housings of different sizes, the shaft housing configured to connect to the connector at a hinge joint that allows for uniplanar rotation to enable alignment adjustments;
selecting an extension shaft from a plurality of extension shafts of different lengths, wherein a fastener secures the extension shaft to the shaft housing for stability; and
selecting a wrist adaptor from a plurality of wrist adaptors, the wrist adaptor configured to attach to the extension shaft;
wherein the shaft housing together and the extension shaft provide an adjustable length of the apparatus between a range of 85 mm and 260 mm.
